(19) United States
(12) Patent Application Publication
(10) Pub. No.: US 2019/0276537 A1
(43) Pub. Date: Sep. 12, 2019

(54) Segregation Modulation for Immunotherapy

(71) Applicant: The Regents of the University of California, Oakland, CA (US)

(72) Inventors: Daniel A. Fletcher, Berkeley, CA (US); Matthew H. Bakalar, Berkeley, CA (US); Eva M. Schmid, Berkeley, CA (US)

(73) Assignee: The Regents of the University of California, Oakland, CA (US)

(21) Appl. No.: 16/414,937

(22) Filed: May 17, 2019

Related U.S. Application Data
(63) Continuation of application No. PCT/US17/63162, filed on Nov. 22, 2017.
(60) Provisional application No. 62/425,504, filed on Nov. 22, 2016.

(51) Int. Cl. C07K 16/28; C07K 16/30; C07K 16/46

(52) U.S. Cl. CPC C07K 16/2803; C07K 16/3007; C07K 16/2818; C07K 2317/55; C07K 16/468; C07K 2317/31; C07K 16/289

(57) ABSTRACT

Segregation modulation therapeutics comprise a recombinant macromolecule comprising an effector-binding module and an anchor-binding module joined by a linker, wherein the modules are specific for an effector and an anchor, respectively of a target cell, and bound thereto, the enforces an effector-anchor complex such that the effector and anchor molecules are spatially co-localized on the target cell.

This invention was made with government support under Grant Numbers GM118190 and GM079465 awarded by the National Institutes of Health. The government has certain rights in the invention.

### INTRODUCTION

Immune cell signaling is associated with defined patterns of spatial organization, such as the segregation of TCR from CD45 in the “immunological synapse” of T-cells. Response to immunotherapeutics is often limited by immune suppression through self-recognition, which is also expected to depend on spatial organization of cell surface molecules such as CD47.

We recently found that small changes in the size of proteins can drive dramatic changes in segregation of molecules at cell-cell contacts. We investigated whether artificially reorganizing cell surface molecules with synthetic macromolecules can change effector cell function. The concept is that immune signaling, as well as that of other cells, can be altered through physical manipulation of receptor organization. Macromolecular therapeutics that modulate the segregation of cell surface molecules, or “Segregation Modulators”, could be used to drive new function and/or confer additional properties to existing immunotherapeutic tools such as antibodies that have high ligand affinity but poor efficacy due to mechanisms of evasion.

Relevant literature: E. M. Schmid, M. H. Bakalar, K. Choudhuri, J. Weichsel, H. S. Ann, P. L. Geissler, M. L. Dustin, and D. A. Fletcher, “Size-dependent protein segregation at membrane interfaces”, Nature Physics (2016) doi:10.1038/nphys3678.

### SUMMARY OF THE INVENTION

This invention describes a set of therapeutic macromolecules that can be used to control cell signaling for immunotherapeutic purposes. The therapeutic macromolecules act by modulating the segregation of cell surface molecules of immune cells or their targets. Composed of one, two or more binding sites connected by linkers of variable size and stiffness, the therapeutic macromolecules (i) selectively connect one, two or more cell surface molecules to each other and/or (ii) alter their relative size and/or affinity. This results in spatial reorganization of cell surface molecules and changes immune effector cell function, causing, for example, a macrophage to phagocytose a tumor cell that it otherwise would not. The therapeutic macromolecules can be added exogenously to cells, generated by a reaction in situ, or produced genetically within a cell so that they act on the extracellular and/or intracellular domains of membrane proteins. In addition to immunotherapeutic applications, the therapeutic macromolecules described here can be used to modulate signaling at a broad range of cell-cell junctions for developmental or wound-healing applications, or they can be used as adjuvants in combination with other therapies.

Accordingly, the invention provides methods and compositions to confer additional properties to existing immunotherapeutic tools such as antibodies that have high ligand affinity but poor efficacy due to mechanisms of evasion.

In an aspect, the invention provides a recombinant bispecific macromolecule or protein comprising an effector-binding module and an anchor-binding module joined by a linker, wherein the modules are specific for an effector and an anchor, respectively of a target cell, and bound thereto, the molecule enforces an effector-anchor complex such that the effector and anchor molecules are spatially co-localized on the target cell.

In embodiments:

the anchor molecule has physical properties, such as height, charge, or affinity for membrane domains that control the spatial localization of the effector-anchor complex, and by doing so prevent productive interaction between the effector protein and a human immune effector cell, disrupting the immune response;

the anchor-binding module comprises a recombinant protein or protein domain, such as an antibody or antibody fragment (e.g. Fab), that binds with high specificity and high affinity to the anchor molecule on the outer surface of a target cell;

the anchor molecule is a protein, peptide, glycan, glycolipid, or lipid, and has physical properties that determine its localization on the cell surface;

the anchor molecule has a large height, such that close apposition of a human immune effector cell forces the molecule outside of the cell-cell interface, the anchor molecule localizes to specialized lipid domains, and/or the anchor molecule is an enzyme, such as a kinase or phosphatase, which can act locally to suppress binding or signaling of the effector molecule;

the effector molecule is a protein, peptide, glycan, glycolipid, lipid, or any composition of these components or their degraded products; and/or

the effector-binding module possess high affinity for the molecule of interest in its native state, or requires a small-molecule or protein cofactor to become competent for binding, such as wherein the target-binding module is activated for binding by the application of visible or infrared light, or by a predetermined range of pH values.

In particular examples, the protein is: (i) bispecific against an immuno-suppressive recognition marker, such as a SIRPα recognition marker like CD47, and cancer biomarker or antigen, like CEACAM5, to suppress cancer cell immuno-evasion; (ii) bispecific against PD-1 and an activating receptor, such as DAP12, on T-cells, to rewire inhibitor signaling into activating signaling; or (iii) bispecific against inhibitory receptor, such as PD-1 or CD47, and CD45, to switch off inhibitory signaling.

In another aspect, the invention provides methods of making the subject proteins and methods of using the subject proteins as an immunotherapy, or to enhance the effectiveness of an immunotherapy.

The invention encompasses all combination of the particular embodiments recited herein, as if each combination had been laboriously recited.

### BRIEF DESCRIPTION OF THE FIGURES

FIGS. 1, 2. Paperclip therapeutic

FIGS. 3, 4. Doorstop therapeutic

FIGS. 5, 6. Step-ladder therapeutic

FIGS. 7, 8. Power-strip therapeutic

FIGS. 9, 10. Paper crease therapeutic

FIGS. 11, 12. Staple therapeutic

### DESCRIPTION OF PARTICULAR EMBODIMENTS OF THE INVENTION

This invention provides a new approach to immunotherapy that modulates the segregation of molecules on immune or other cells to alter their interaction, providing a method for preventing tumor cells from escaping immune recognition or modulating autoimmune reactions. Clinical applications include pathologies that depend on the organization of cell surface molecules for signaling and downstream function, including cancer, autoimmune diseases, inflammatory diseases, neurodegenerative diseases, developmental diseases, immunizations, parasitic infections, etc.

Related technologies include antibodies that direct immune effector function to target cells bearing specific molecules through an Fc effector domain; however, promiscuous expression of cell-surface proteins across both healthy and diseased cell types lowers the specificity of the immune response, resulting in autoimmunity. Blocking antibody fragments (Fabs) prevent binding to specific molecules on target cells, but do not engage immune effector cells to promote a broad immune response. Bispecific antibodies direct immune effector cells to target cells by ligating a target molecule to an immune cell-surface protein; similar to antibodies, promiscuous expression of cell-surface proteins across both healthy and diseased cell types can lead to mistargeting and autoimmunity.

The Segregation Modulation therapeutics use antibodies or other protein binding domains, together with a linker domain, to modify the spatial organization or physical conformation of molecules on a target cell, enabling a therapeutically useful immune response from effector immune cells. This approach is more specific than existing bispecific antibodies that directly recruit an immune effector cell. Instead of recruiting an effector cell directly, it promotes a spatial arrangement of molecules on a target cell that prevents a target cell from evading the immune response, allowing native immune effector mechanisms to engage with the target cell.

Below we describe a preferred embodiment of the Segregation Modulation therapeutics followed by multiple alternate embodiments. For convenience, we have named the different embodiments after common office supplies that loosely relate to the activity of the therapeutic macromolecule described.

Paperclip therapeutic. This is a bispecific macromolecule that binds two molecules with different physical heights, such that the spatial organization that arises during interaction between a target cell and an immune effector cell is altered.

The first portion of the therapeutic protein binds to an effector molecule located on a target cell, which typically interacts with a human immune effector cell to enhance or suppress an immune response. The second portion of the protein binds to an anchor molecule located on the same target cell. The protein enforces an effector-anchor complex such that the effector and anchor molecules are spatially co-localized on the target cell. The anchor molecule has physical properties, such as height, charge, or affinity for membrane domains that control the spatial localization of the effector-anchor complex, and by doing so prevent productive interaction between the effector protein and a human immune effector cell, disrupting the immune response.

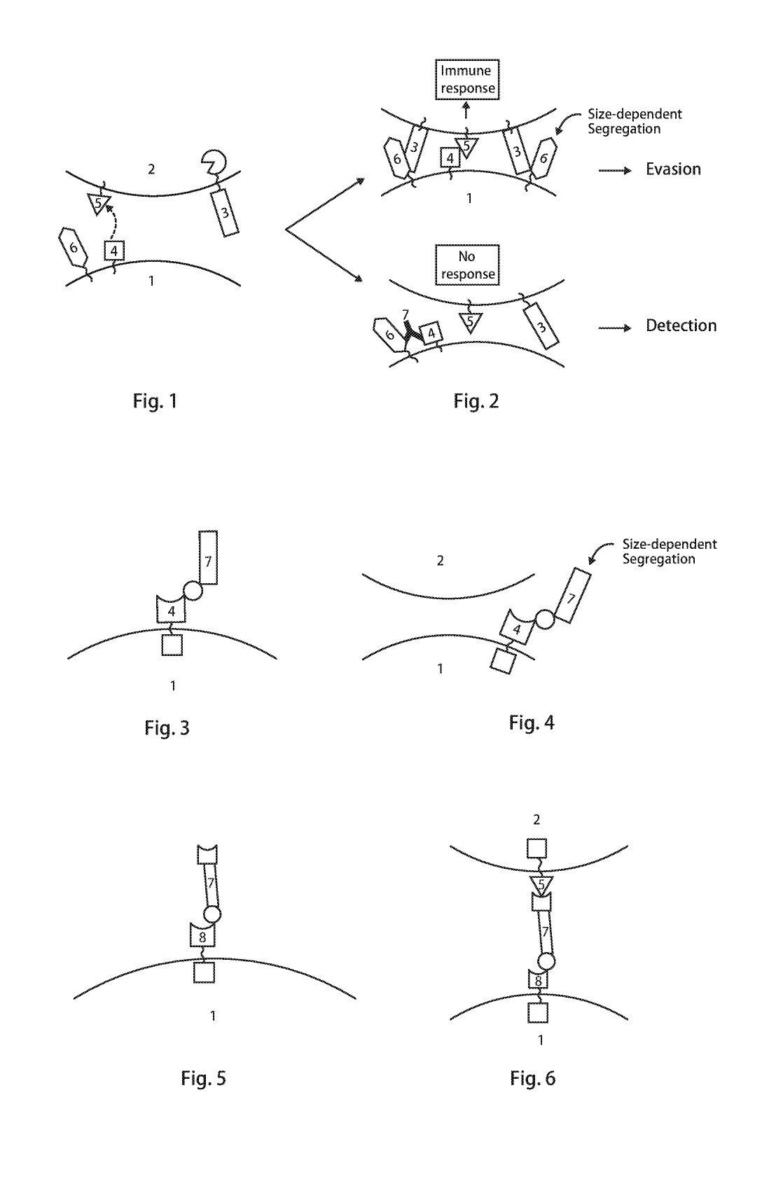

FIGS. 1-2 show a target cell 1, effector cell 2, anchor binding module 3, effector target molecule 4, effector binding module 5, anchor molecule 6 and bispecific therapeutic 7.

These proteins are composed of an effector-binding module, which may consist of a recombinant protein or protein domain, including an antibody or antibody fragment. This effector-binding module can bind with high specificity and high affinity to an effector molecule on the outer surface of a target cell. The effector molecule can be a protein, peptide, glycan, glycolipid, lipid, or any composition of these components or their degraded products. The effector-binding module can possess high affinity for the molecule of interest in its native state, or it may require a small-molecule or protein cofactor to become competent for binding. In one embodiment, the target-binding module is activated for binding by the application of visible or infrared light. In another embodiment, the target-binding module is activated for binding by a particular range of pH values. In another embodiment, the target-binding module is activated for binding by temperature changes, such as during a fever or application of localized heating.

The effector-binding module is connected to the anchor-binding module by a polypeptide or polymer linker. The linker may be composed of one or more amino acids or polymer monomers, so that it spans a prescribed length from a few angstroms to tens of micrometers. In one embodiment, the linker can be cleaved by an endogenous or exogenous enzyme. In another embodiment, the linker length can be increased or decreased by binding to a small-molecule or protein cofactor.

The anchor-binding module may consist of a recombinant protein or protein domain, including an antibody or antibody fragment. This anchor-binding module binds with high specificity and high affinity to an anchor molecule on the outer surface of a target cell. An anchor molecule can be a protein, peptide, glycan, glycolipid, or lipid. The anchor molecule has physical properties that determine its localization on the cell surface. In one preferred embodiment, the anchor molecule has a large height, such that close apposition of a human immune effector cell forces the molecule outside of the cell-cell interface. In another embodiment, the anchor molecule localizes to specialized lipid domains. In another embodiment, the anchor molecule is an enzyme, such as a kinase or phosphatase, which can act locally to suppress binding or signaling of the effector molecule.

As one specific example, the effector molecule could be the human protein CD47, and the anchor molecule could be the human colon cancer antigen CEACAM5. In this example, the effector and anchor targeting modules consist of a bispecific antibody without Fc effector activity. One Fab would target CD47, and the second Fab would target the membrane-proximal domain of CEACAM5, causing CD47 and CEACAM5 to segregate together on the cell surface. On normal cells, CD47 is displayed on the surface of cells as a marker of self. Typically, interaction between CD47 and the macrophage receptor SIRPα sends a “don't eat me” signal to macrophage immune effector cells. However, this mechanism is hijacked by cancer cells to evade immune detection. Signaling by SIRPα requires physical segregation of molecules on the cell-surface of the immune effector cell, where large phosphatases such as CD45 and CD148 are excluded from the site of contact with the target cell. A large target-specific anchor-molecule connects the spatial localization of CD47 to that of CEACAM5, disrupting the ability of CD47-SIRPα complex to segregate from large phosphatases, a process that is required for productive signaling. This therapeutic approach will overcome the ability of cancer cells expressing CD47 to evade recognition by macrophages.

Paperclip chain therapeutic. In this embodiment three or more binding interactions are designed to cluster specific molecules. The first portion of the therapeutic protein binds to an anchor molecule located on a target cell. The second portion of the protein comprises multivalent dendrimer or binding domains, which may have the same or different specificities for effector molecules on the target cell. This reagent enforces a one-to-many spatial relationship between the anchor molecule and one or more types of effector molecules on the target cell.

Doorstop therapeutic. This embodiment changes the size of a molecule by binding an inert domain with a defined size and shape to an effector molecule.

FIGS. 3-4 show a target cell 1, effector cell 2, effector target molecule 4, and bispecific therapeutic 7.

In this embodiment, the first portion of the therapeutic protein binds to an effector molecule located on a target cell, which typically interacts with a human immune effector cell to enhance or suppress an immune response. The second portion of the protein comprises a protein module with a particular height or steric volume, which when bound to the effector molecule has physical properties that prevent productive interaction between the effector molecule and a human immune effector cell by modifying the spatial organization of the effector molecule through size-dependent physical segregation, disrupting the native immune response. In addition to the single binding domain therapeutic described above, a bifunctional therapeutic could have the same effect of size-dependent physical segregation if it binds to a large soluble serum protein that does not bind to immune effector molecules.

Step-ladder therapeutic. This embodiment extends the size of a molecule while maintaining binding affinity, or changing binding specificity through an adaptor.

FIGS. 5-6 show a target cell 1, effector cell 2, bispecific therapeutic 7, anchor molecule 8, and effector binding module 5.

In this embodiment, the first portion of the therapeutic protein binds to an anchor molecule located on a target cell. The second portion of the protein comprises a protein domain that extends the height of the molecule perpendicular to the membrane. The third portion of the protein comprises an effector domain that binds to a protein on the surface of a human immune effector cell, which may include the original binding partner of the anchor molecule or a new protein on the immune effector cell. When bound to the anchor molecule this reagent changes the effective height of the interaction between target cell immune effector cell, modulating an immune response against the target cell.

Power-strip therapeutic. This embodiment alters organization by linking an effector domain with a membrane-binding domain.

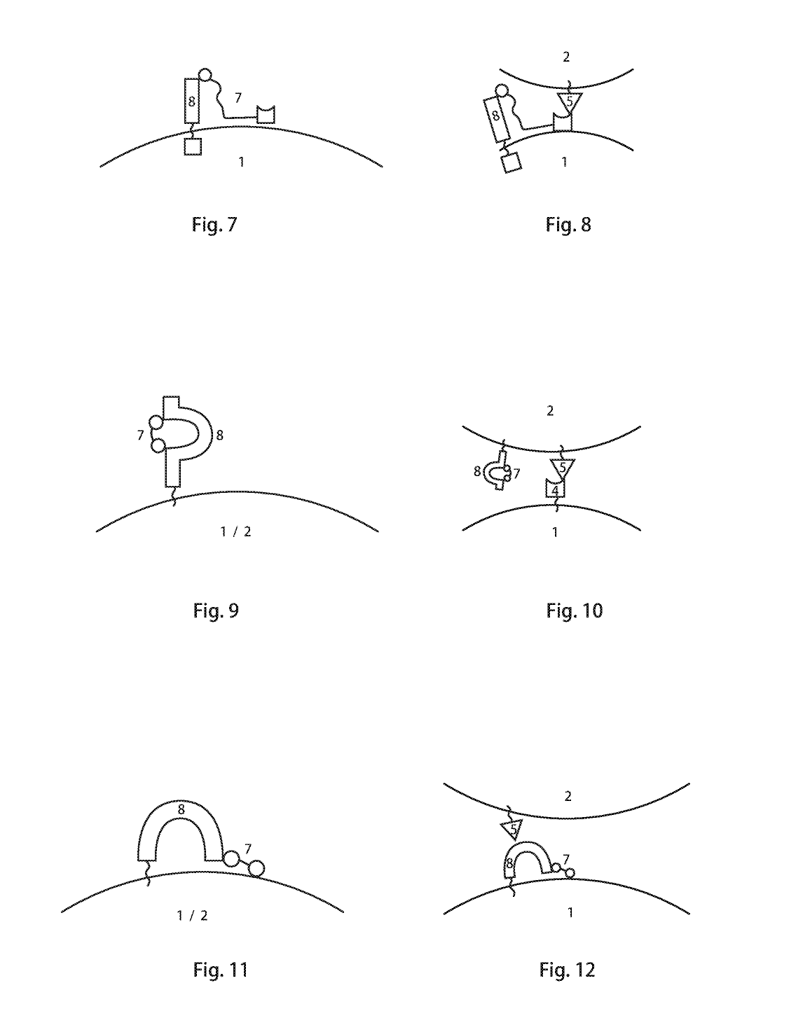

FIGS. 7-8 show a target cell 1, effector cell 2, bispecific therapeutic 7, anchor molecule 8, and effector binding module 5.

The first portion of the protein binds to an anchor molecule located on a target cell. The second portion of the protein comprises an effector domain that binds to a protein on the surface of a human immune effector cell. The effector domain is attached via a flexible linker that also binds to the membrane of the target cell, forcing the effector domain into close proximity with the surface of the target cell where it can productively segregate proteins from the contact interface between a human immune effector cell and a target cell to produce an immune response.

Paper crease therapeutic. This embodiment decreases the size of a molecule while maintaining native binding affinity by internally binding two or more parts of it to change conformation.

FIG. 9-10 show a target cell 1, effector cell 2, effector target molecule 4, effector binding module 5, bispecific therapeutic 7 and effector target molecule 8,

In this embodiment, the first portion of the therapeutic protein binds to a specific site within an effector molecule located on a target cell. The second portion of the protein comprises a protein module that binds to a separate site within the effector molecule. These modules are connected by a small linker domain. The reagent has the effect of folding the conformation of an effector molecule, decreasing its height without modifying its binding affinity.

Staple therapeutic. This embodiment decreases the size of a molecule by altering conformation by bending it onto the membrane, either maintaining or altering binding affinity.

FIGS. 11-12 show a target cell 1, effector cell 2, effector binding module 5, bispecific therapeutic 7 and effector target molecule 8,

In this embodiment, the first portion of the therapeutic protein binds to a specific site within an effector molecule located on a target cell. The second portion of the protein comprises a protein module that binds to the membrane of a target cell. These modules are connected by a small linker domain. The reagent has the effect of grabbing on to an effector molecule and bending it down towards the membrane, changing the height of the effector molecule without changing its binding affinity.

Remote-control therapeutic. This embodiment dynamically change binding affinity or linker length of protein modules through small-molecules, pH, light, enzyme activity, temperature, etc.

In alternate embodiments of each of the above embodiments, the effector and anchor binding modules possess high affinity for the molecule of interest in their native state, or require a small-molecule or protein cofactor to become competent for binding. In one embodiment, the target-binding module is activated for binding by the application of visible or infrared light. In another embodiment, the target-binding module is activated for binding by a particular range of pH values. The linker can also be modified dynamically—in one embodiment, the linker can be cleaved by an endogenous or exogenous enzyme. In another embodiment, the linker length can be increased or decreased by binding to a small-molecule or protein cofactor. It should also be noted that altered signaling by segregation modulation therapeutics need not depend on the presence of a cell-cell interface but could also be generated by clustering of receptors away from a cell-cell interface.

Proof of Principle Experiments

We reconstituted model membrane interfaces in vitro using giant unilamellar vesicles decorated with synthetic binding and non-binding proteins. We showed that size differences between membrane proteins can alter their organization at membrane interfaces, e.g. a 5 nm increase in non-binding protein size driving its exclusion from the interface. Combining in vitro measurements with Monte Carlo simulations, we demonstrated that non-binding protein exclusion is also controllable by lateral crowding, binding protein affinity, and thermally driven membrane height fluctuations that transiently limit access to the interface.

Unless contraindicated or noted otherwise, in these descriptions and throughout this specification, the terms “a” and “an” mean one or more, the term “or” means and/or and polynucleotide sequences are understood to encompass opposite strands as well as alternative backbones described herein.

The examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

1. A method of altering cell signaling by modulating the segregation of cell surface molecules of an immune cell or a target cell of the immune cell with a recombinant macromolecule comprising an effector-binding module and an anchor-binding module joined by a linker, wherein the modules are specific for an effector molecule and an anchor molecule, respectively of the target cell, and bound thereto, the method comprising: contacting the target cell with the macromolecule under conditions wherein the macromolecule binds the effector and anchor molecules and thereby enforces an effector-anchor complex such that the effector and anchor molecules are spatially co- localized on the target cell, and thereby alters interaction with the immune cell and immune cell signaling.

2. The method of claim 1 wherein the anchor-binding module comprises a recombinant protein or protein domain that binds with specificity and affinity to the anchor molecule on the outer surface of a target cell, or requires a small-molecule or protein cofactor to become competent for binding, and the effector-binding module comprises a recombinant protein or protein domain that binds with specificity and affinity to the effector molecule on the outer surface of a target cell, or requires a small-molecule or protein cofactor to become competent for binding.

3. The method of claim 1 wherein the effector-binding module requires a small-molecule or protein cofactor to become competent for binding, wherein the target-binding module is activated for binding by the application of visible or infrared light, or by a predetermined range of pH values.

4. The method of claim 1 wherein the anchor-binding module comprises an antibody or antibody fragment (e.g. Fab) specific for the anchor molecule, and the effector-binding module comprises an antibody or antibody fragment (e.g. Fab) specific for the effector molecule.

5. The method of claim 1 wherein a bispecific antibody comprises the anchor-binding and effector-binding modules.

6. The method of claim 1 wherein the macromolecule is bispecific against: (i) an immuno-suppressive recognition marker, and cancer biomarker or antigen, to suppress cancer cell immuno-evasion ; (ii) PD-1 and an activating receptor on T-cells, to rewire inhibitor signaling into activating signaling; or (iii) an inhibitory receptor, and CD45, to switch off inhibitory signaling.

7. The method of claim 5 wherein the macromolecule is bispecific against: (i) an immuno-suppressive recognition marker, and cancer biomarker or antigen, to suppress cancer cell immuno-evasion ; (ii) PD-1 and an activating receptor on T-cells, to rewire inhibitor signaling into activating signaling; or (iii) an inhibitory receptor, and CD45, to switch off inhibitory signaling.

8. The method of claim 1 wherein the macromolecule is bispecific against: (i) CD47 and CEACAM5; (ii) PD-1 and DAP12; or (iii) PD-1 or CD47 and CD45.

9. The method of claim 5 wherein the macromolecule is bispecific against: (i) CD47 and CEACAM5; (ii) PD-1 and DAP12; or (iii) PD-1 or CD47 and CD45.

10. The method of claim 1 wherein the anchor molecule has a physical property selected from height, charge, and affinity for membrane domains that controls the spatial localization of the effector-anchor complex, and thereby prevents productive interaction between the effector protein and a human immune effector cell, disrupting an otherwise resultant immune response.

11. The method of claim 1 wherein the anchor molecule has a height, wherein close apposition of a human immune effector cell forces the molecule outside of the cell-cell interface, the anchor molecule localizes to specialized lipid domains, and/or the anchor molecule is an enzyme which acts locally to suppress binding or signaling of the effector molecule.

12. The method of claim 1 wherein the macromolecule binds two molecules with different physical heights, wherein the spatial organization that arises during interaction between the target cell and the immune effector cell is altered.

13. The method of claim 1 wherein the macromolecule binds to an anchor molecule located on the target cell, and a plurality of effector molecules, and thereby enforces a one-to-many spatial relationship between the anchor molecule and one or more types of effector molecules on the target cell.

14. The method of claim 1 wherein the macromolecule binds to an effector molecule located on a target cell, which interacts with a human immune effector cell to generate a native immune response, and the anchor-binding module comprises a particular height or steric volume, which when bound to the effector molecule has physical properties that prevent productive interaction between the effector molecule and the immune cell by modifying the spatial organization of the effector molecule through size-dependent physical segregation, altering the native immune response.

15. The method of claim 1 wherein the macromolecule binds to an anchor molecule located on a target cell, and to an effector molecule on the surface of the human immune cell, wherein the linker is elongate and flexible and configured to bind to the membrane of the target cell, forcing the effector domain into close proximity with the surface of the target cell where it productively segregates proteins from the contact interface between the immune effector cell and a target cell to produce an immune response.

16. The method of claim 1 wherein the macromolecule binds to a specific site within the effector molecule located on a target cell, and to a separate site within the effector molecule, wherein the modules are connected by a small linker domain, whereby the macromolecule has the effect of folding the conformation of the effector molecule, decreasing its height without modifying its binding affinity.

17. The method of claim 1 wherein the macromolecule binds to a specific site within an effector molecule located on a target cell and binds to the membrane of a target cell, whereby the macromolecule has the effect of grabbing on to an effector molecule and bending it down towards the membrane, changing the height of the effector molecule without changing its binding affinity.

18. The method of claim 1 wherein the macromolecule is added exogenously to the target or immune cell.

19. The method of claim 1 wherein the macromolecule is expressed by the target or immune cell.

20. A recombinant macromolecule comprising an effector-binding module and an anchor-binding module joined by a linker, wherein the modules are specific for an effector molecule and an anchor molecule, respectively of a target cell, and bound thereto, the macromolecule is configured to enforce an effector-anchor complex such that the effector and anchor molecules are spatially co-localized on the target cell, wherein optionally:
(a) the anchor molecule has a physical property selected from height, charge, and affinity for membrane domains that controls the spatial localization of the effector-anchor complex, and thereby prevents productive interaction between the effector protein and a human immune effector cell, disrupting an otherwise resultant immune response;
(b) the anchor-binding module comprises a recombinant protein or protein domain that binds with specificity and affinity to the anchor molecule on the outer surface of a target cell;
(c) the anchor molecule is a protein, peptide, glycan, glycolipid, or lipid, and has predetermined physical properties to configure its localization on the cell surface;
(d) the anchor molecule has a height, wherein close apposition of a human immune effector cell forces the molecule outside of the cell-cell interface, the anchor molecule localizes to specialized lipid domains, and/or the anchor molecule is an enzyme which acts locally to suppress binding or signaling of the effector molecule;
(e) the effector molecule is a protein, peptide, glycan, glycolipid, lipid, or any compositions or combinations or degradation products thereof;
(f) the effector-binding module possesses high affinity for the molecule of interest in its native state, or requires a small-molecule or protein cofactor to become competent for binding;
(g) the effector-binding module requires a small-molecule or protein cofactor to become competent for binding, wherein the target-binding module is activated for binding by the application of visible or infrared light, or by a predetermined range of pH values;
(h) the macromolecule is bispecific (i) an immuno-suppressive recognition marker, and cancer biomarker or antigen, to suppress cancer cell immuno-evasion ; (ii) PD-1 and an activating receptor on T-cells, to rewire inhibitor signaling into activating signaling; or (iii) inhibitory receptor, and CD45, to switch off inhibitory signaling; and/or
(i) the macromolecule is bispecific against (i) CD47 and CEACAM5; (ii) PD-1 and DAP12; or (iii) PD-1 or CD47 and CD45 or a method of using the macromolecule as an immunotherapy, or to enhance the effectiveness of an immunotherapy.
